(19) United States
(12) Patent Application Publication
(10) Pub. No.: US 2026/0060543 A1
(43) Pub. Date: Mar. 5, 2026

(54) METHOD FOR RELAYING ECG DATA

(71) Applicants: Kuang-Hsing Chiang, Taipei City (TW); Jih Yin, Taipei City (TW)

(72) Inventors: Kuang-Hsing Chiang, Taipei City (TW); Jih Yin, Taipei City (TW)

(21) Appl. No.: 19/315,423

(22) Filed: Aug. 29, 2025

(30) Foreign Application Priority Data
Aug. 29, 2024 (TW) 113132513

(51) Int. Cl. A61B 5/00

(52) U.S. Cl. CPC A61B 5/0006

(57) ABSTRACT

A method for relaying ECG data is disclosed. The method may selectively transmit part of the ECG data from an ECG detection device to a medical information system server through a mobile communication device. The method may comprise steps of continuously receiving ECG data detected by and sent from the ECG detection device; synchronously calculating heart rate variability corresponding to each consecutive time point of the ECG data; and when a calculated heart rate variability is lower than a threshold, sending all ECG data in a corresponding time period to the medical information system server, until the calculated heart rate variability is not lower than the threshold.

### CROSS-REFERENCE TO RELATED APPLICATION

This patent application claims the benefit of Intellectual Property Office Ministry Of Economic Affairs Republic Of China Application Number 113132513, entitled Method For Relaying ECG Data,” filed Aug. 29, 2024, which application is incorporated in its entirety here by this reference.

### TECHNICAL FIELD

This invention relates to a method for relaying data. More particularly, the present invention relates to a method for relaying ECG data.

### BACKGROUND

Electrocardiogram (ECG) is a method of recording the electrical activity of the heart and is primarily used to diagnose and monitor heart diseases. In the last century, when measuring devices could not be miniaturized, patients with heart diseases had to go to the hospital in person for testing during treatment. In addition to the high medical expenses, the time or conditions required to detect the cause of the heart disease are unpredictable, causing patients to waste a lot of medical time. With the miniaturization of electronic devices, the size of ECG measuring devices has also become smaller. ECG measuring devices can even transmit the measured data wirelessly to a remote data collection center at any time, such as the server of a medical information system, so that doctors can read the patient's ECG data remotely in real time and provide treatment services to patients as soon as symptoms occur, thereby protecting patients'healthy lives.

Functions of existing portable ECG measuring devices are very powerful. In addition to being able to detect a full range of data, such as basic ECG parameters (such as P wave, PR interval, QRS complex, ST segment, T wave and QT interval), heart rate and rhythm, the detected data can also be used to further calculate derived parameters, such as heart rate variability (HRV) and late potentials. The portable ECG measuring devices can completely transmit these parameters to the data collection center through a relay device, such as a smartphone; some portable ECG measuring devices with Internet access can also transmit data directly. The function of the portable ECG measuring device to transmit data completely is not only to consider the integrity of the data, but also because the storage space of the portable ECG measuring device is limited and the detected ECG data cannot be permanently retained. However, most of the data transmitted by the portable ECG measuring devices to the data collection center are the patient's ECG data under normal conditions, and very few of them are useful for doctors to determine the cause of heart-related diseases. While the data collection center continues to collect ECG data, it also has to spend some of its resources analyzing the data to find out the parts that are relevant to judging the patient's condition, which is a waste of medical resources. Preferably, the ECG data transmitted by the portable ECG measuring device is valid data that can be directly provided to doctors for diagnosis of the diseases. However, the portable ECG measuring devices currently available on the market do not meet the above needs.

### SUMMARY

The summary of the invention set forth below is intended as a description of presently preferred embodiments of the invention and is not intended to represent the only forms in which the present invention may be constructed or utilized. It is to be understood, however, that the same or equivalent functions and sequences may be accomplished by different embodiments that are also intended to be encompassed within the spirit and scope of the invention.

The present invention is directed to a system and method for identification and relay of valid ECG data that is relevant to the diagnosis of one or more diseases of a patient. In order to solve the above problems, a method for relaying ECG data is disclosed in the present invention. In some embodiments, the method of the present invention may selectively transmit part of ECG data from an ECG detection device to a medical information system server through a mobile communication device. In certain embodiments, the system and method may comprise the steps of: continuously receiving ECG data detected by and sent from the ECG detection device; synchronously calculating heart rate variability at each consecutive time point corresponding to the ECG data; when calculated heart rate variability is lower than a relevant threshold, transmitting ECG data in a corresponding time period to the medical information system server until the calculated heart rate variability is not lower than the threshold.

In some embodiments, the system and method of the present invention may selectively transmit part or a portion of ECG data from an ECG detection device to a medical information system server through a mobile communication device. In certain embodiments, the system and method may comprise the steps of: continuously receiving ECG data detected by the ECG detection device and heart rate variability of each consecutive period of the ECG data in time series sent from the ECG detection device; and when the heart rate variability is lower than a threshold, transmitting all ECG data in a corresponding time period to the medical information system server, until the calculated heart rate variability is not lower than a threshold.

In some embodiments, the system and method of the present invention may selectively transmit part or a portion of ECG data from an ECG detection device to a medical information system server through a mobile communication device. In certain embodiments, the system and method may comprise the steps of: continuously receiving ECG data detected by and sent from the ECG detection device; if a trigger start signal from the ECG detection device is received, calculating an absolute value of difference between a highest value and a lowest value of the heart rate variability in a judgment period before and after the trigger start signal is sent, and in many embodiments if, for example, the absolute value of difference is greater than a trigger value, transmitting the ECG data detected after the trigger start signal is sent to a medical information system server; and if a trigger stop signal from the ECG detection device is received or after a transmission time has passed, stopping the transmission of the ECG data to the medical information system server. In some embodiments an additional step may be included if the absolute value of difference is not greater than the trigger value but the same trigger start signal is received for the second time within an emergency time limit, transmitting the ECG data detected after the first trigger start signal is sent to the medical information system server. According to many embodiments of the present invention, from the time the trigger start signal may first be sent to the time the trigger stop signal is sent or to the time after the transmission time has passed, the mobile communication device may fetche ECG data at a certain sampling rate and transmit the fetched ECG data to the medical information system server.

In some embodiments, the method for relaying ECG data may selectively transmit part or a portion of ECG data from an ECG detection device to a medical information system server through a mobile communication device. Such a method may comprise the steps of: continuously receiving ECG data detected by and sent from the ECG detection device; synchronously calculating heart rate variability at each consecutive time point corresponding to the ECG data; and when calculated heart rate variability is higher than a threshold, transmitting all ECG data in a corresponding time period to the medical information system server, until the calculated heart rate variability is not higher than the threshold.

In some embodiments, the method for relaying ECG data may selectively transmit part or a portion of ECG data from an ECG detection device to a medical information system server through a mobile communication device. Such a method may comprise the steps of: continuously receiving ECG data detected by the ECG detection device and heart rate variability of each consecutive period of the ECG data in time series sent from the ECG detection device; and when the heart rate variability is higher than a threshold, transmitting all ECG data in a corresponding time period to the medical information system server, until the calculated heart rate variability is not higher than the threshold.

According to many embodiments of the present invention, the trigger start signal and the trigger stop signal may be sent by a detected person operating the ECG detection device. The trigger start signal and the trigger stop signal may also be sent by the ECG detection device after the ECG detection device detects a physical condition from itself. The physical condition may be rapid flipping, rapid falling, or external environment temperature higher than normal human body temperature.

According to many embodiments of the present invention, the mobile communication device may be a smartphone, a tablet or a smart wearable device.

In some embodiments, a computer program, such as software, source code, compiled code, or other forms of programs that run on a computing device may be embodied as part of the present invention. Such a program may be installed in a mobile communication device or other computing device, operating to perform the method for relaying ECG data disclosed above.

The present invention may use a mobile communication device or alternative computing or network device as a relay device between an ECG detection device and a medical information system server to control the ECG data transmitted from the ECG detection device to the medical information system server. Compared with conventional electrocardiogram measuring devices, the present invention selectively transmits portions of ECG data for medical staff to determine or diagnose a patient's current condition under certain conditions, without providing the complete ECG data to the medical information system server. On one hand, it reduces the load on the medical information system server, and on the other hand, it allows medical staff to more quickly and effectively understand the patient's current condition and provide immediate diagnosis and treatment and thus solve the aforementioned problems.

### BRIEF DESCRIPTION OF DRAWINGS

FIG. 1 shows a hardware architecture for executing a method for relaying ECG data of the present invention;

FIG. 2 shows a flowchart of an embodiment of the method for relaying ECG data of the present invention;

FIG. 3 shows an ECG with calculated heart rate variability listed on the ECG;

FIG. 4 shows a flowchart of an embodiment of a method for relaying ECG data of the present invention;

FIG. 5 shows a flowchart of a third embodiment of the method for relaying ECG data of the present invention; and

FIG. 6 is a flowchart of a fourth embodiment of the method for relaying ECG data of the present invention.

### DETAILED DESCRIPTION OF THE INVENTION

The detailed description set forth below in connection with the appended drawings is intended as a description of presently preferred embodiments of the invention and is not intended to represent the only forms in which the present invention may be constructed or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

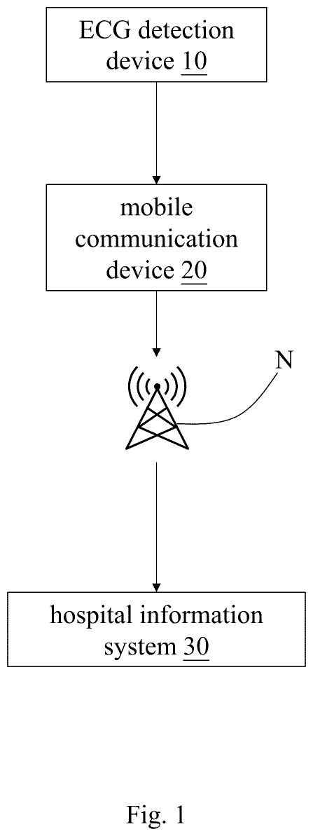

Referring to FIG. 1, the invention comprises a hardware architecture for executing a method for relaying ECG data of the present invention. Basically, the hardware architecture includes an ECG detection device 10, a mobile communication device 20 and a medical information system server 30. According to the present invention, the type of the ECG detection device 10 is not limited. It may be fixed or portable, but it must be able to transmit data wirelessly, detect the aforementioned basic ECG parameters, heart rate, rhythm, and calculate derived parameters. The mobile communication device 20, may be but not limited to a smartphone, a tablet or a smart wearable device. The mobile communication device 20 can connect to the USB module, Wi-Fi module or Bluetooth module of the ECG detection device 10 through its USB module, Wi-Fi module or Bluetooth module, so as to receive the ECG data from the ECG detection device 10. As a data transmission relay device, the mobile communication device 20 can selectively transmit part of the ECG data to the medical information system server 30 through the network N. “Selectively” means that the mobile communication device 20 can transmit useful part of the ECG data (abnormal condition) to the medical information system server 30, and the remaining ECG data (normal condition) can be directly discarded, and all ECG data do not need to be stored in the mobile communication device 20. Here, the network N can be any network architecture for data transmission, such as fixed network or mobile communication network. In order to implement the method for relaying ECG data, a computer application program (APP) may be installed in the mobile communication device 20. When the computer application program is executed on the mobile communication device 20, the specific steps of the method for relaying ECG data in many embodiments of the present invention may be executed.

Additionally, or in the alternative, the term computer application program may refer to or include software such as one or more processes, one or more instances, Application Programming Interface(s) (API), subroutine(s), function(s), applet(s), servlet(s), routine(s), source code, object code, shared library/dynamic link library (DLL), or even one or more instructions. Such software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

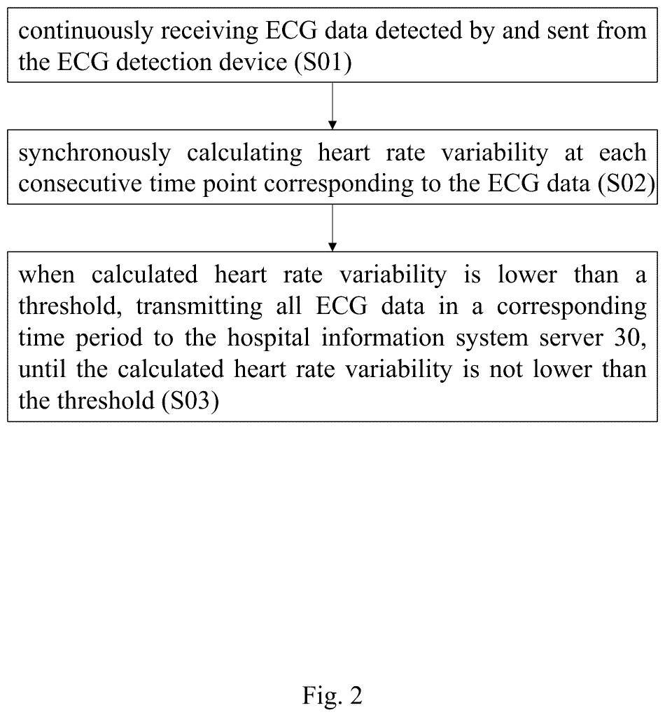

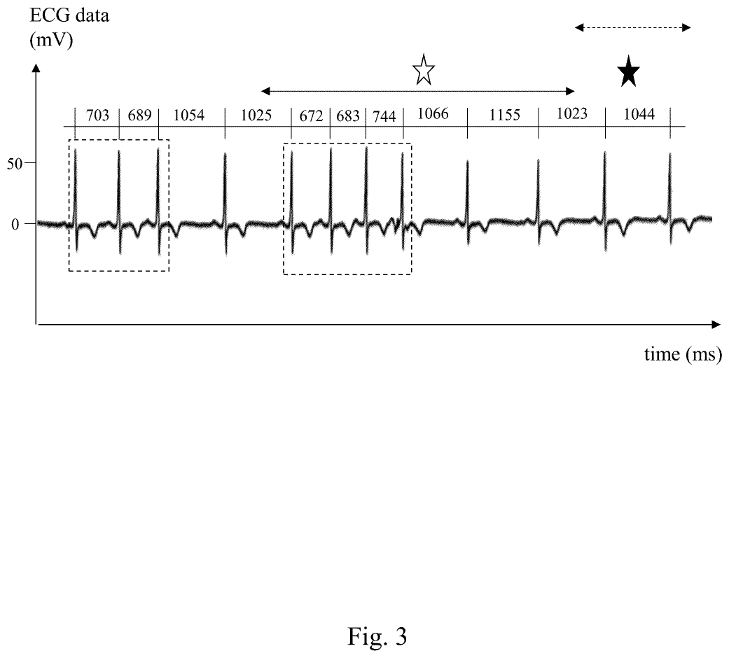

As shown in the flow diagram of FIG. 2, in some embodiments of the system and method for relaying ECG data of the present invention, a first step of the method for relaying ECG data may continuously receive ECG data detected by and be sent from the ECG detection device 10 (S01). A second step may comprise synchronously calculating heart rate variability at each consecutive time point corresponding to the ECG data (S02). In order to have a better understanding of this, FIG. 3 shows, for example, an embodiment where an ECG calculates heart rate variability listed on the ECG. The vertical axis value of the ECG depicts the ECG data, which represents the voltage value (mV) detected by the ECG detection device 10. The horizontal axis of the electrocardiogram represents time, in milliseconds. Heart rate variability refers to the time difference between each consecutive heartbeat, also known as “R-R intervals variation”. Simply put, everyone's heartbeat rhythm changes with their physical condition, rather than being as regular as a beat machine. For example, a person's resting heart beats about 60 times per minute, but the interval between each beat is actually different. As shown in FIG. 3, in many embodiments, the time between the P wave peaks of two adjacent heartbeats is used as the calculation basis, and the number above the ECG is the heart rate variability.

An additional step of the method for relaying ECG data in the certain embodiments may be when calculated heart rate variability is lower than a threshold, transmitting all ECG data in a corresponding time period to the medical information system server 30, until the calculated heart rate variability is not lower than the threshold (S03). By way of example, 800 mS is set as the threshold. Hence, in FIG. 3, the ECG data corresponding to the heart rate variability of 703 mS, 689 mS, 672 mS, 683 mS and 744 mS (indicated by the dotted line frame) may be transmitted from the mobile communication device 20 to the medical information system 30. The other parts of ECG data that are not lower than the threshold may be discarded and not used by the mobile communication device 20. It should be noted that along with the ECG data sent to the medical information system 30, there may also be other data obtained from the mobile communication device 20, such as the current time, temperature, humidity, location, etc., which can be used by doctors to assist in making judgments when viewing the ECG data. In practice, step S03 may be changed to “when calculated heart rate variability is higher than a threshold, transmitting all ECG data in a corresponding time period to the medical information system server 30, until the calculated heart rate variability is not higher than the threshold”. At this time, the mobile communication device 20 uses a higher heart rate variability as a benchmark for triggering the transmitting of ECG data. The threshold can be selected as the lower or upper limit of the trigger according to the medical staff's judgment of the patient's condition.

An additional step of the method for relaying ECG data in certain embodiments is when calculated heart rate variability is lower than a threshold, transmitting all ECG data in a corresponding time period to the medical information system server 30, until the calculated heart rate variability is not lower than the threshold (S03). In some embodiments, 800 mS is set as the threshold. Hence, in FIG. 3, the ECG data corresponding to the heart rate variability of 703 mS, 689 mS, 672 mS, 683 mS and 744 mS (indicated by the dotted line frame) may be transmitted from the mobile communication device 20 to the medical information system 30. The other parts of ECG data that are not lower than the threshold may be discarded and not used by the mobile communication device 20. It should be noted that along with the ECG data sent to the medical information system 30, there may also be other data obtained from the mobile communication device 20, such as the current time, temperature, humidity, location, etc., which can be used by doctors to assist in making judgments when viewing the ECG data. In practice, step S03 may be changed to “when calculated heart rate variability is higher than a threshold, transmitting all ECG data in a corresponding time period to the medical information system server 30, until the calculated heart rate variability is not higher than the threshold.” At this time, the mobile communication device 20 may use a higher heart rate variability as a benchmark for triggering the transmitting of ECG data. The threshold can be selected as the lower or upper limit of the trigger according to a medical staff member's judgment of a patient's condition.

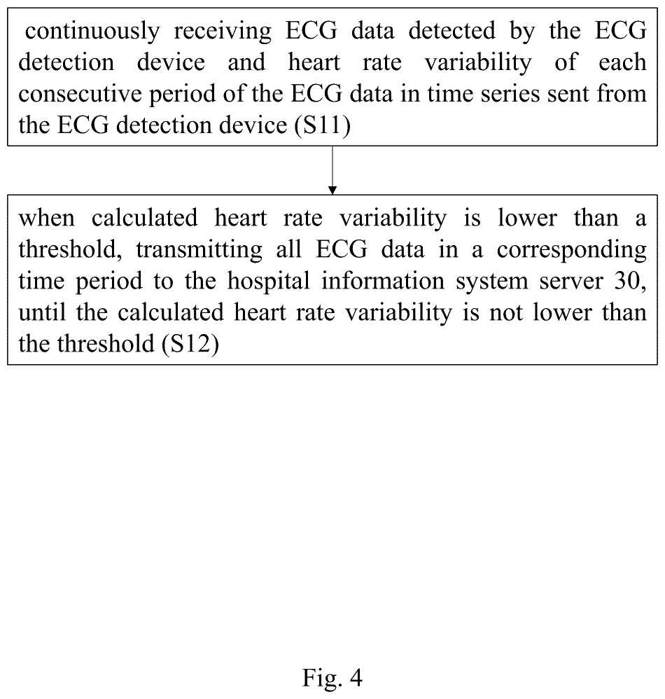

As illustrated in the flow diagram shown in FIG. 4, some embodiments of the method for relaying ECG data of the present invention may include a step of the method for relaying ECG data for continuously receiving ECG data detected by an ECG detection device 10 and heart rate variability of each consecutive period of the ECG data in time series sent from the ECG detection device 10 (S11). In some embodiments, the ECG detection device 10 can calculate the heart rate variability and send it to the mobile communication device 20 along with the ECG data. Therefore, the mobile communication device 20 does not need to calculate the heart rate variability additionally. Further, an additional step of the method for relaying ECG data in some embodiments may be calculated when heart rate variability is lower than a threshold, transmitting ECG data in a corresponding time period to the medical information system server 30, until the calculated heart rate variability is not lower than the threshold (S12). Step S12 may be the same as step S03 of the previous embodiments, and its technical content may not be repeated. Similar to the previous embodiments, step S12 can also be changed to “when the heart rate variability is higher than a threshold, transmitting all ECG data in a corresponding time period to the medical information system server 30, until the calculated heart rate variability is not higher than the threshold.” At this time, the mobile communication device 20 also may use a higher heart rate variability as a benchmark for triggering the transmitting of the ECG data.

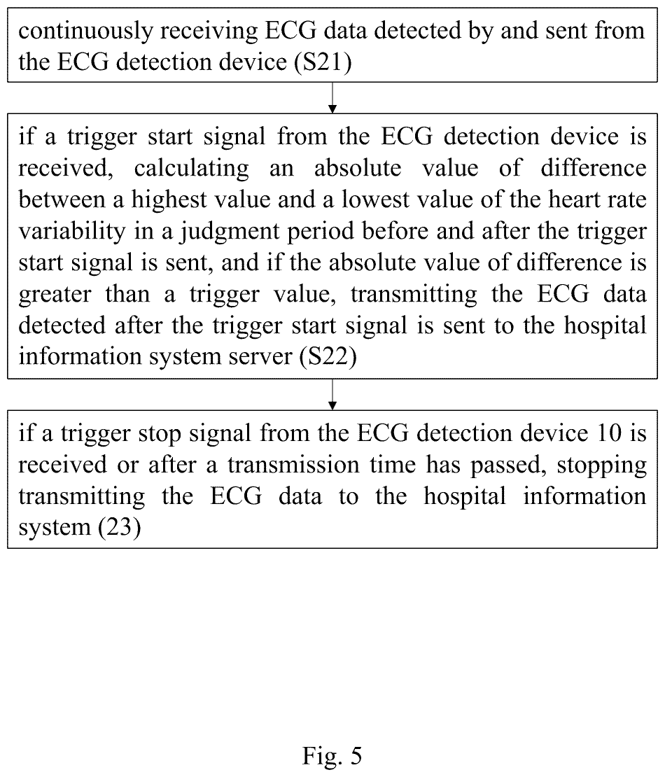

As illustrated in the flow diagram shown in FIG. 5, an embodiment of the method for relaying ECG data of the present invention may comprise a method for relaying ECG data where certain embodiments are continuously receiving ECG data detected by and sent from the ECG detection device 10 (S21). An additional step may occur if a trigger start signal from the ECG detection device 10 is received, calculating an absolute value of difference between a highest value and a lowest value of the heart rate variability in a judgment period before and after the trigger start signal is sent, and if the absolute value of difference is greater than a trigger value, transmitting the ECG data detected after the trigger start signal is sent to the medical information system server 30 (S22). To illustrate step S22, referring to FIG. 3 again, in some embodiments, the judgment period may be set as 3 seconds before and after (total duration 6 seconds) the trigger start signal is sent. In practice, it may not be limited to this and can be set according to the characteristics of the patient. In FIG. 3, the time when the trigger start signal is sent is indicated by a hollow asterisk, which may be based on the actual time of the ECG detection device 10 to synchronize with the timing of the ECG. In the judgment period, 1025 mS, 672 mS, 683 mS, 744 mS, 1066 mS, 1155 mS and 1023 mS may be obtained as the heart rate variability (the judgment period is represented by a solid double arrow). Among them, the highest value is 1155 mS and the lowest value is 672 mS. Hence, the absolute value of difference is 482 mS. If the trigger value is set as 400 mS, then the mobile communication device 20 transmits the ECG data after the hollow asterisk to the medical information system 30. On the contrary, for example, if the trigger value is set as 500 mS, the absolute value of difference is smaller than the trigger value. Then, the mobile communication device 20 does not transmit the ECG data to the medical information system 30. An additional step of the method for relaying ECG data in some embodiments may occur if a trigger stop signal from the ECG detection device 10 is received or after a transmission time has passed, stopping transmitting the ECG data to the medical information system server 30 (S23). Said “transmitting the ECG data to the medical information system server 30” will not last. The stop time may be the time when the trigger stop signal is sent by the ECG detection device 10, and the time is indicated by a solid asterisk in FIG. 3. Hence, the ECG data actually received by the medical information system server 30 falls within the time points indicated by the hollow asterisk and the solid asterisk. Often, the detected person (patient) forgets to send the trigger stop signal after sending the trigger start signal, causing the mobile communication device 20 to continue to send invalid ECG data (in a healthy state). To solve this problem, the present invention uses a computer application program to set the transmission time, such as 10 minutes. After the transmission time has passed since the ECG data was transmitted to the medical information system server 30, the transmission of the ECG data is automatically stopped.

In some embodiments of the method for relaying ECG data of the present invention, a step S22′ may be further included after step S22: if the absolute value of difference is not greater than the trigger value but the same trigger start signal is received for the second time within an emergency time limit, transmitting the ECG data detected after the first trigger start signal is sent to the medical information system server 30. Sometimes, although the computer application program of the mobile communication device 20 determines that there is no need to transmit the ECG data, the patient still continues to feel uncomfortable and sends a trigger start signal through the ECG detection device 10 again. If the time interval between the two trigger start signals is very long, such as 1 hour, the mobile communication device 20 will start again from step S21 to determine whether to transmit the ECG data through the control of the computer application program. However, if the time interval between the two trigger start signals is very short, it means that the patient may have real symptoms, and the ECG data should be uploaded to the medical information system server 30 as soon as possible for medical staff to diagnose. In response to this delayed physiological discomfort, the present embodiment no longer determines whether to transmit the ECG data but directly transmits the ECG data after the first trigger start signal is sent to the medical information system server 30, until the trigger stop signal (in this case, it is not limited by the transmission time) is sent. Said emergency time limit may be the maximum allowed time between two trigger start signals when performing direct ECG data transmission, for example 30 seconds.

According to the present invention, from the time the trigger start signal is first sent to the time the trigger stop signal is sent, or from the time the trigger start signal is first sent to the time after the transmission time has passed, the mobile communication device 20 may fetch the ECG data at a sampling rate automatically and synchronously with the previous step, and transmits the fetched ECG data to the medical information system server 30. In some embodiments, the present invention does not limit the sampling rate, and the sampling rate may be once every 30 seconds or once every minute. The purpose of the mobile communication device 20 performing this operation is to sample the ECG data that may be normal in order to compare it with the ECG data that may be problematic obtained in steps S21 to S23. For example, within the 1-hour transmission time after the trigger start signal is first sent, the problematic ECG data may be sent from the 3rd to the 6th minute, and from the 23rd to the 37th minute. The ECG data from the beginning to the 3rd minute, from the 6th minute to the 23rd minute, and from the 37th minute to the 60th minute are determined by the computer application program to be possibly normal ECG data and are not sent to the medical information system server 30. For the medical staffs, if there is some possibly normal ECG data to refer to (heart rate change trend), then they can further confirm the symptoms behind the potentially problematic ECG data they received. As mentioned above, the present invention aims to avoid transmitting all ECG data to the medical information system server 30 at one time, which would waste resources. Therefore, by using the fixed frequency sampling technology within the aforementioned transmission time, medical staffs can obtain some “discrete” ECG data of patients under normal circumstances. When medical staffs observe the potentially problematic continuous ECG data from the 3rd to 6th minute and the 23rd to 37th minute, there may also be some auxiliary data points that can help show the trend of heart rate changes. Of course, these “discrete” ECG data sampled at the fixed frequency will also obtain the same data as the 3rd to 6th minutes and the 23rd to 37th minutes, which does not need to be deliberately excluded.

According to many embodiments of the present invention, the trigger start signal and trigger stop signal are generated by the detected person (patient) operating the ECG detection device 10. The purpose is to allow the patient to promptly instruct the mobile communication device 20 to transmit the ECG data within a certain period of time to the medical information system server 30 through the ECG detection device 10 when feeling unwell, so that medical staffs can determine how to provide emergency treatment measures to the remote patient based on this urgent information. Of course, sometimes the patient feels uncomfortable for other reasons that have nothing to do with the heart's response. The absolute value of difference is the basis for the mobile communication device 20 to determine whether to transmit the ECG data. As mentioned above, the length of the judgment period can be set according to the characteristics of the patient. This is the time it takes for the patient to feel uncomfortable and then send out a trigger start signal. Elderly people or patients with limited mobility are inevitably slow in operating the ECG detection device 10. A longer judgment period can allow the mobile communication device 20 to have more heart rate variability data for reference. In addition, the trigger start signal and the trigger stop signal can also be sent by the ECG detection device 10 after detecting a physical condition of itself (the ECG detection device 10). The physical condition may be rapid flipping (according to a built-in acceleration sensor of the ECG detection device 10), rapid falling (according to the built-in acceleration sensor of ECG detection device 10), or external environment temperature higher than normal human body temperature (according to a thermometer built into the ECG detection device 10). In addition to the patient's voluntary request to send ECG data, in the event of some sudden conditions of the patient, such as falling down, falling out of bed, or being in an external dangerous environment without knowing it, such as being in 39 degrees Celsius outdoor, the ECG detection device 10 can also autonomously let the mobile communication device 20 transmit ECG data to the medical information system server 30 to avoid sudden heart disease.

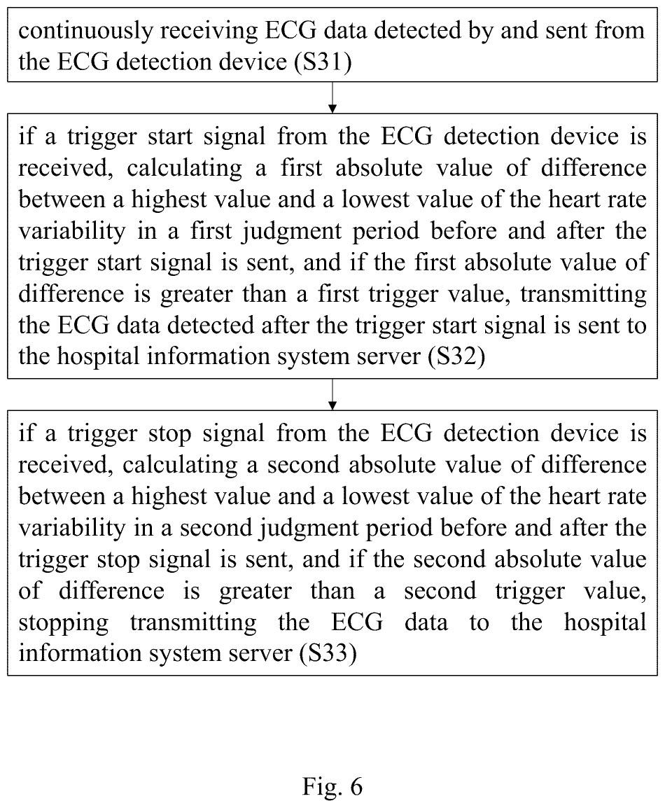

As illustrated in the flow diagram of FIG. 6, some embodiments of the method for relaying ECG data of the present invention, a step may include continuously receiving ECG data detected by and sent from an ECG detection device 10 (S31). A second step is if a trigger start signal from the ECG detection device 10 is received, calculating a first absolute value of difference between a highest value and a lowest value of the heart rate variability in a first judgment period before and after the trigger start signal is sent, and if the first absolute value of difference is greater than a first trigger value, transmitting the ECG data detected after the trigger start signal is sent to the medical information system server 30 (S32). This step may be substantially the same as step S22 of the previous embodiments, except that the aforementioned judgment period may called the first judgment period to distinguish it from a second judgment period in the next step; the aforementioned absolute value of difference is called the first absolute value of difference to distinguish it from a second absolute value of difference in the next step; and the aforementioned trigger value is called the first trigger value to distinguish it from a second trigger value in the next step. Next, a step of the method for relaying ECG data, in some embodiments, may be a trigger stop signal from the ECG detection device 10 is received, calculating a second absolute value of difference between a highest value and a lowest value of the heart rate variability in a second judgment period before and after the trigger stop signal is sent, and if the second absolute value of difference is greater than a second trigger value, stopping transmitting the ECG data to the medical information system server (S33). Obviously, step S33 is not the same as step S23 of the previous embodiment. After receiving the trigger stop signal from the ECG detection device 10, the mobile communication device 20 stops transmitting ECG data to the medical information system server 30. In some embodiments, the mobile communication device 20 performs a similar operation as that performed upon receiving a trigger stop signal to determine whether to stop transmitting the ECG data. In some embodiments, assume that the second judgment period is 1 second before and after the trigger stop signal is sent (total duration 2 seconds). In practice, it is not limited to this and can also be set according to the characteristics of the patient. In FIG. 3, the time when the trigger stop signal is issued is marked with a solid asterisk. In a second judgment period, 1023 mS and 1044 mS may be obtained as the heart rate variability (the second judgment period is represented by a dotted double arrow). The second absolute value of difference is 21 mS. If the second trigger value is set to 100 mS, in the example of FIG. 3, the mobile communication device 20 may continue to transmit ECG data to the medical information system server 30 even if it receives the trigger stop signal. This is to prevent the patient from operating the ECG detection device 10 incorrectly and accidentally shutting down the ECG data transmission, which would cause an emergency and no relevant data for the doctor to diagnose.

According to another aspect of the present invention, there is provided a non-transitory computer-readable medium including executable instructions that, when executed with one or more processors, cause the ECG data relay system and method to perform any of the methods as described above.

Similar to some previous embodiments, in many embodiments, the trigger start signal and the trigger stop signal can be sent by the detected person (patient) operating the ECG detection device 10. In addition, the trigger start signal and the trigger stop signal may also be sent after the ECG detection device 10 detects a physical condition, such as rapid flipping, rapid falling, or external environment temperature higher than normal human body temperature, of itself.

The foregoing description of the preferred embodiments of the invention has been presented for the purposes of illustration and description. It is not intended to be exhaustive or to limit the invention to the precise form disclosed. Many modifications and variations are possible in light of the above teachings. It is intended that the scope of the invention not be limited by this detailed description, but by the claims and the equivalents to the claims appended hereto.

What is claimed is:

1. A method for relaying electrocardiogram (ECG) data, for selectively transmitting part of ECG data from an ECG detection device to a medical information system server through a mobile communication device, the method comprising:
a. continuously receiving ECG data detected by and sent from the ECG detection device;
b. synchronously calculating a heart rate variability at each consecutive time point corresponding to the ECG data; and
c. when a calculated heart rate variability is lower than a threshold, transmitting the ECG data in a corresponding time period to the medical information system server until the calculated heart rate variability is not lower than the threshold.

2. The method for relaying ECG data according to claim 1, wherein the mobile communication device is a smartphone, a tablet, or a wearable computing device.

3. The method for relaying ECG data according to claim 1, the method to further comprising:
a. receiving a trigger start signal from the ECG detection device;
b. calculating an absolute value of difference between a highest value and a lowest value of the heart rate variability in a judgment period before and after the trigger start signal is sent;
c. transmitting the ECG data detected after the trigger start signal is sent to the medical information system server if the absolute value of difference is greater than a trigger value; and
d. stopping transmitting the ECG data to the medical information system server if the trigger stop signal from the ECG detection device is received or after a transmission time has passed.

4. The method for relaying ECG data according to claim 3, the method further comprising:
transmitting the ECG data detected after the first trigger start signal is sent to the medical information system server if the absolute value of difference is not greater than the trigger value but the same trigger start signal is received for the second time within an emergency time limit.

5. The method for relaying ECG data according to claim 4, wherein from the time the trigger start signal is first sent to the time the trigger stop signal is sent or to the time after the transmission time has passed, the mobile communication device fetches the ECG data at a sampling rate and transmits the fetched ECG data to the medical information system server.

6. The method for relaying ECG data according to claim 3, wherein the trigger start signal and the trigger stop signal are sent by a detected person operating the ECG detection device.

7. The method for relaying ECG data according to claim 3, wherein the trigger start signal and the trigger stop signal are sent by the ECG detection device after the ECG detection device detects a physical condition thereof.

8. The method for relaying ECG data according to claim 7, wherein the physical condition is rapid flipping, rapid falling, or external environment temperature higher than normal human body temperature.

9. The method for relaying ECG data according to claim 4, wherein the trigger start signal and the trigger stop signal are sent by a detected person operating the ECG detection device.

10. The method for relaying ECG data according to claim 4, wherein the trigger start signal and the trigger stop signal are sent by the ECG detection device after the ECG detection device detects a physical condition thereof.

11. The method for relaying ECG data according to claim 10, wherein the physical condition is rapid flipping, rapid falling, or external environment temperature higher than normal human body temperature.

12. The method for relaying ECG data according to claim 5, wherein the trigger start signal and the trigger stop signal are sent by a detected person operating the ECG detection device.

13. The method for relaying ECG data according to claim 5, wherein the trigger start signal and the trigger stop signal are sent by the ECG detection device after the ECG detection device detects a physical condition thereof.

14. The method for relaying ECG data according to claim 13, wherein the physical condition is rapid flipping, rapid falling, or external environment temperature higher than normal human body temperature.

15. The method for relaying ECG data according to claim 3, wherein the mobile communication device is a smartphone, a tablet or a smart wearable device.

16. A method for relaying ECG data for selectively transmitting a portion of ECG data from an ECG detection device to a medical information system server through a mobile communication device, the method comprising:
a. continuously receiving ECG data detected by and sent from the ECG detection device;
b. synchronously calculating heart rate variability at each consecutive time point corresponding to the ECG data; and
c. when calculated heart rate variability is higher than a threshold, transmitting all ECG data in a corresponding time period to the medical information system server, until the calculated heart rate variability is not higher than the threshold.

17. The method for relaying ECG data according to claim 16, wherein the mobile communication device is a smartphone, a tablet or a smart wearable device.

18. A method for relaying ECG data, for selectively transmitting part of ECG data from an ECG detection device to a medical information system server through a mobile communication device, the method comprising:
a. continuously receiving ECG data detected by the ECG detection device and heart rate variability of each consecutive period of the ECG data in time series sent from the ECG detection device; and
b. when the heart rate variability is higher than a threshold, transmitting all ECG data in a corresponding time period to the medical information system server, until the calculated heart rate variability is not higher than the threshold.

19. A computer program product, installed in the mobile communication device, operating to perform the method for relaying ECG data of any one of claims 1-18.

20. A non-transitory computer-readable medium including executable instructions that, when executed with one or more processors, cause the computer program product to perform the method for relaying ECG data of any one of claims 1-19.
